(19) United States
(12) Patent Application Publication
(10) Pub. No.: US 2025/0154446 A1
(43) Pub. Date: May 15, 2025

(54) MAGNET-DRIVEN BISTABLE DYNAMIC BIOREACTOR

(71) Applicant: City University of Hong Kong Shenzhen Futian Research Institute, Shenzhen (CN)

(72) Inventors: Jian LU, Shenzhen (CN); Zhou CHEN, Shenzhen (CN); Chengshengze CHU, Shenzhen (CN); Yunhu HE, Shenzhen (CN); Shiting CHEN, Shenzhen (CN); Wanying WANG, Shenzhen (CN); Chong WANG, Shenzhen (CN)

(21) Appl. No.: 18/825,400

(22) Filed: Sep. 5, 2024

(30) Foreign Application Priority Data
Nov. 14, 2023 (CN) 202311518215.8

(51) Int. Cl. C12M 1/42; B33Y 80/00; C12M 1/06; C12M 1/12; C12M 1/34; C12M 3/00

(52) U.S. Cl. CPC C12M 35/06; B33Y 80/00; C12M 23/06; C12M 23/48; C12M 27/06; C12M 41/12

(57) ABSTRACT

A magnet-driven bistable dynamic bioreactor, its manufacturing method, and application thereof are provided. The bioreactor has a magnet-driven bistable actuator, a magnetic field generation module, and a connected temperature control module. The magnet-driven bistable actuator includes a magnetic bistable arch membrane. The magnetic field generation module provides a direction-alternating magnetic field, which drives the magnetic bistable arch membrane to switch states between different configurations. This process applies adjustable frequency biaxial bi-directional loading on cells that cultured on membrane, simulating real tissue loading. The temperature control module regulates the temperature of the magnet-driven bistable actuator. The bioreactor enables adjustable frequency biaxial bi-directional loading on cells cultured on the magnetic bistable arch membrane by utilizing an alternating magnetic field to switch its configuration. This process effectively simulates the loading environment experienced by cells in real tissues.

### CROSS-REFERENCE TO RELATED APPLICATIONS

The present application claims priority based on the prior application No. 20231151 8215.8, submitted to the China National Intellectual Property Administration on Nov. 14, 2023, entitled ‘A magnet-driven bistable dynamic bioreactor’. The entire content of the prior application is incorporated herein by reference.

### TECHNICAL FIELD

The present disclosure belongs to the field of biomechanics, and specifically, relates to a magnetic control bistable cellular mechanical loading device and manufacturing method and application thereof.

### BACKGROUND

With the advancement of biomechanics and tissue engineering, the study of cell growth and proliferation behavior has become a research focus in biomedical engineering. In vitro cell loading experiments not only simplify the loading conditions for cells but also more effectively simulate the mechanical behavior of cells, indicating the influence on cell function, growth, and differentiation under dynamic loading.

Currently, there are various methods and platforms for in vitro cell loading experiments, with the most common method involving the stretching or compression of the culture substrate, resulting in strains acting on the cells. Tensile loading is applied to cells by inducing tension deformation of the elastic substrate through pneumatic, hydraulic, or mechanical devices. Conversely, compression loading is achieved by using a mechanical device to compress the culture substrate. Although the aforementioned methods broadly conform to the characteristics of cellular stress conditions in tissue engineering, they have limitations due to the single loading mode and the fixed amount of substrate deformation, leading to uneven membrane stress as well as uneven cellular stress. Additionally, fatigue and fracture of the elastic substrate are primary factors affecting the reliability and robustness of the existing methods. Crucially, cells in tissues (e.g. cardiomyocytes, small intestinal endothelial cells, smooth muscle cells) undergo complex stress in most cases, such as biaxial (transverse and longitudinal) and bidirectional (tension and compression) loads. Therefore, existing uniaxial tensile devices are unable to accurately simulate the real stress state of cells.

Furthermore, the currently available commercial bioreactors mostly rely on stretching and squeezing. It is challenging to adjust the loading frequency throughout the experiment due to the relatively singular loading mode. Meanwhile, the instrument requires high maintenance fee, and the working condition of the entire platform is unstable. Some custom-made cell loading platforms are more complex and less universally applicable than those commercially available. Currently, there is a lack of research and development on biaxial and bidirectional loading devices recently.

### SUMMARY

To address the inadequacies of current technology, this disclosure presents a magnet-driven bistable dynamic bioreactor along with relevant manufacturing methods and their applications. To simulate the loading conditions in real tissues and study the mechanical response behavior of cells, this dynamic bioreactor is based on a magnetic bistable arch membrane that can switch its configuration between two different states upon the application of an alternating magnetic field. This approach resolves issues related to single loading mode, unadjustable frequency, poor versatility, and low-test efficiency. Importantly, it achieves biaxial and bidirectional loading at an adjustable frequency.

Specifically, the present disclosure provides the following technical solutions:

A magnet-driven bistable dynamic bioreactor comprises a temperature control module, a magnetic field generation module, and a magnet-driven bistable actuator placed in a Petri dish. The magnet-driven bistable actuator comprises a magnetic bistable arch membrane. The temperature control module regulates the temperature of the bistable actuator. The magnetic field generation module applies a direction-alternating magnetic field to the bistable actuator, driving the magnetic bistable arch membrane to switch its configuration between different states. This enables adjustable frequency biaxial and bidirectional loading of cells cultured on membrane to mimic real tissue loading condition. In one embodiment of the present disclosure, a fixed periphery and a bracket frame are additional components of the magnet-driven bistable actuator. The bonding interlayers are introduced between the magnetic bistable arch membrane and fixed periphery and the bracket frame, respectively. It features a hole adapted to fit the outer contour of the magnetic bistable arch membrane, with the hole partially or completely bonding to the outer contour to constrain the rigid body displacement of the magnetic bistable arch membrane.

In one embodiment of the present disclosure, the support columns bond to the lower end of the bracket frame. The fixed periphery solely constrains the rigid body displacement of the magnetic bistable arch membrane, and the combination of the bracket frame and support columns enables the magnetic bistable arch membrane to be suspended in the cell culture medium.

In one embodiment of the present disclosure, the magnetic bistable arch membrane is a three-dimensional curved structure and serves as a substrate for cell culture.

In one embodiment of the present disclosure, the magnetization orientation of the bistable arch membrane is perpendicular to the tangent line of the membrane surface and introduces an angle between the external applied magnetic field. The misalignment between magnetization profile and magnetic field induces a magnetic torque to the bistable arch membrane. The magnetic bistable arch membrane switches from the concave state to the convex state by the magnetic torque. The configuration transition process can be reversed by changing the direction of the magnetic field.

In one embodiment of the present disclosure, the magnetic field generation module consists of a current control unit, an electromagnet, and a power source. The module, which provides a magnetic field with alternating changes in magnetic direction, switches the bistable arch membrane configuration. The magnet-driven bistable actuator is positioned on the top of the electromagnet's pole, and an alternating current is generated under the control of the current control unit. The alternating current excites the electromagnet to generate the direction-alternating magnetic field.

Preferably, the magnet-driven bistable actuator, Petri dish, and electromagnet are placed in a carbon dioxide cell culture incubator.

In one embodiment of the present disclosure, the temperature control module comprises a control circuit and a cooling system, which includes a coolant tank, a condensate tube, and a temperature sensor. The temperature sensor detects temperature changes in the cell culture medium surrounding the magnet-driven bistable actuator and transmits the temperature signals to the control circuit. The control circuit activates the cooling system to regulate the temperature of the electromagnet based on the temperature sensor signal. The condensate tube surrounds the electromagnet, and both the electromagnet and the condensate tube are placed in a carbon dioxide cell culture incubator. The control circuit is used for regulating the flow rate of condensate in the condenser tube. In one embodiment of the present disclosure, the magnetic bistable arch membrane is made of biocompatible material and magnetic particles.

In one embodiment of the present disclosure, the biocompatible material comprises one or more of polydimethylsiloxane (PDMS), polyvinyl alcohol (PVA), polycaprolactone (PCL), acylated gelatin methacrylate (GelMa), polyethylene terephthalate (PET), polyurethane (PU) and et. al.

In one embodiment of the present disclosure, the magnetic particles comprise one or more of rubidium iron boron magnetic particles, aluminum nickel cobalt magnetic particles, hard ferrite magnetic particles, and samarium cobalt hard magnetic particles.

The present disclosure also provides a method for preparing the magnet-driven bistable dynamic bioreactor, which includes preparing the magnet-driven bistable actuator and connecting it to the temperature control module and the magnetic field generation unit. The method is outlined as follows:
1. Prepare a magnetic bistable arch membrane containing magnetic particles by casting;
2. Reorient the magnetization direction of the magnetic particles embedded in the bistable arch membrane;
3. Prepare a fixed periphery with a hole adapted to the magnetic bistable arch membrane by 3D printing, and bond the magnetic bistable arch membrane to the fixed periphery;
4. Attach the fixed periphery and magnetic bistable arch membrane combination to the bracket frame and construct a support column to ensure the stable placement of the combination in the cell culture medium.

In one embodiment of the present disclosure, the method comprises:
1) Inject a magnetic prepolymer mixture into a mold with an arch cavity using the pouring method, and prepare a magnetic bistable arch membrane by performing the steps of degassing, drying and demolding;
2) Magnetize the magnetic bistable arch membrane by applying an externally magnetic field based on the hysteresis loop property of the magnetic particles. After magnetization, the orientation of the magnetic particles in the arch membrane is perpendicular to the tangent line of the arch membrane. Finally, we obtain a magnetic bistable arch membrane with oriented magnetization;
3) Prepare a fixed periphery by 3D printing, and bond the magnetic bistable arch membrane to the fixed periphery partially or entirely to constraint the magnetic bistable arch membrane;
4) Prepare a bracket frame and establish a permanent interlayer bond between the bracket frame and the fixed periphery;
5) Bond a support column to the bracket frame to ensure the stable placement of the magnetic bistable arch membrane in the cell culture medium.

In one embodiment of the present disclosure, the step 1) comprises:
Step 11) Mix the base liquid and crosslinking agent in a specific mass ratio to prepare the prepolymer mixture, then mix the prepolymer mixture with magnetic particles in a specific volume ratio to obtain the magnetic prepolymer mixture;
Step 12) Pour the magnetic prepolymer mixture into the mold, and degas under negative pressure circumstances and ensure uniform distribution of prepolymer mixture in the mold;
Step 13) Place the entire casting mold in the oven for drying and curing for a period. Demold the magnetic bistable arch membrane after cooling.

In one embodiment of the present disclosure, said step 2) comprises:
Step 21) Attach the demolded magnetic bistable arch membrane to a clean glass, cover it with another piece of glass and press tightly, ensuring that the magnetic bistable arch membrane adheres to the glass consistently and smoothly;
Step 22) Vertically press the assembled magnetic bistable arch membrane and the glass plate into the bottom of the magnetizer chamber, and maintain the compression load until the magnetization process is completed;
Step 23) By applying an external magnetic field larger than the coercivity of the magnetic particles, re-magnetize the magnetic particles to form a uniformly oriented magnetization pattern perpendicular to the membrane plane. The applied magnetic field should exceed the saturation magnetization of the magnetic particles;
Step 24) Remove the magnetic bistable arch membrane and the glass plate from the bottom of the magnetizer chamber, and peel the arch membrane from the glass plate. After peeling, the magnetic bistable arch membrane recovers to its bending state, with the magnetization direction perpendicular to the tangent direction of the membrane surface.

In one embodiment of the present disclosure, the fixed periphery is prepared using the additive manufacture method in step 3), comprises:
Step 31) Mix the base liquid and crosslinking agent in some mass ratio thoroughly to prepare the 3D printing ink;
Step 32) Print a fixed periphery using as-prepared 3D printing ink, there is a hole in the fixed periphery whose diameter is equal to that of the magnetic bistable arch membrane;
Step 33) Bond the magnetic bistable arch membrane partially or fully along the outer circumference to the as-printed uncured fixed periphery, and then cure the combination in an oven at a certain temperature.

In one embodiment of the present disclosure, the step 4) includes:
Step 41) Mix the base liquid and crosslinking agent fully and uniformly at a certain mass ratio, and degas to obtain a prepolymer mixture;
Step 42) Pour the prepolymer mixture into the mold cavity, place the mold in negative circumstance to remove air bubbles, ensuring the even distribution of the prepolymer mixture. This 3D cylindrical mold has a base with the same dimensions as the fixed periphery;
Step 43) Place the mold and the prepolymer mixture into an oven for drying and curing; Step 44) Bond the fixed periphery to the bracket frame with a permanent interlayer using UV Ozone plasma technology.

In one embodiment of the present disclosure, the mass ratio of the base liquid and cross-linking agent varies from 20:1 to 5:1.

The present disclosure also demonstrates applications of the magnet-driven bistable dynamic bioreactor in mimicking the loading conditions of human cells, such as human dermal fibroblasts, human epidermal keratinized cells, renal distal tubular epithelial cells, human cardiomyocytes, osteoblasts, and others.

The beneficial effects of the present invention:
1) The present disclosure describes a simple, frequency-adjustable, biaxial, and bidirectional magnet-driven bistable loading device, which consists of a magnet-driven bistable actuator with a magnet-sensitive bistable arch membrane. The arch membrane is made of soft biocompatible polymer, and its deflection is adjustable to provide culture surface with different curvatures.
2) Upon the application of a direction-alternating magnetic field, the magnetic bistable arch membrane snaps between two states. The snapping behavior yields a frequency-adjustable biaxial bidirectional stress field on the surface of the membrane. This loading device is particularly suitable for studying the mechanical response of cells to dynamic loading, including cells' growth, differentiation, protein expression, and pathological changes.
3) The magnet-sensitive bistable arch membrane provided by the present invention has two stable configurations without external load. These two stable configurations can switch between each other upon the application of a magnetic field. The deflection of the magnetically bistable arched membrane is adjustable, and its dimension are compatible with the existing high-throughput cell culture vessels, thereby enhancing the versatility of the bioreactor.

### BRIEF DESCRIPTION OF THE ATTACHED DRAWINGS

FIG. 1: The schematic diagram of the magnet-driven bistable dynamic bioreactor of the present disclosure.

FIG. 2: The schematic diagram of the magnet-driven bistable actuator, the Petri dish, and the magnetic field generation module of the present disclosure.

FIG. 3: The schematic diagram of the direction-alternating magnetic field generated by the magnetic field generation module.

FIG. 4: The cross-sectional diagram of the magnetic-sensitive bistable arch membrane.

FIG. 5: The strain distribution schematic diagram of the magnetic-sensitive bistable arch membrane.

FIG. 6: The schematic diagram for the first embodiment of the magnet-driven bistable actuator.

FIG. 7: The schematic diagram for the second embodiment of the magnet-driven bistable actuator.

FIG. 8: The radial strain on the surface of the magnetic-sensitive bistable arch membrane during the snapping process.

FIG. 9: The circumferential strain on the surface of the magnetic-sensitive bistable arch membrane during the snapping process.

FIG. 10: Fluorescent images of human dermal fibroblasts (HDFa) form different analysis zones along the circle with a radius of 8 mm (white dot line) in the strain distribution map for the experimental group.

FIG. 11: Fluorescent images of human dermal fibroblasts (HDFa) form different analysis zones along the circle with a radius of 8 mm (white dot line) in the strain distribution map for the control group.

Numbered items in figures:

| | |
|---|---|
| 1 | Magnet-driven bistable actuator |
| 101 | Magnet-sensitive bistable arch membrane |
| 102 | Fixed periphery |
| 103 | Bracket frame |
| 104 | The first support column |
| 105 | The second support column |
| 201 | Electromagnet |
| 202 | Current control unit |
| 203 | Power supplier |
| 301 | Temperature sensor |
| 302 | Condenser tube |
| 303 | Coolant tank |
| 304 | Control circuit |

### DETAILED DESCRIPTION

As previously described, the present disclosure introduces a magnet-driven bistable dynamic bioreactor. The loading device consists of a magnet-driven bistable actuator 1, a magnetic field generation module, and a temperature control module. Specifically, the magnet-driven bistable actuator is composed of a magnet-sensitive bistable arch membrane 101, a fixed periphery 102, and a bracket frame 103. The magnetic field generation module applies a direction-alternating magnetic field to the magnet-driven bistable actuator, driving the bistable arch membrane to snap between two different configurations. The resulting bending deformation of the membrane applies biaxial and bidirectional loading on the cells cultured on the membrane surface, mimicking the real stress conditions of the tissue.

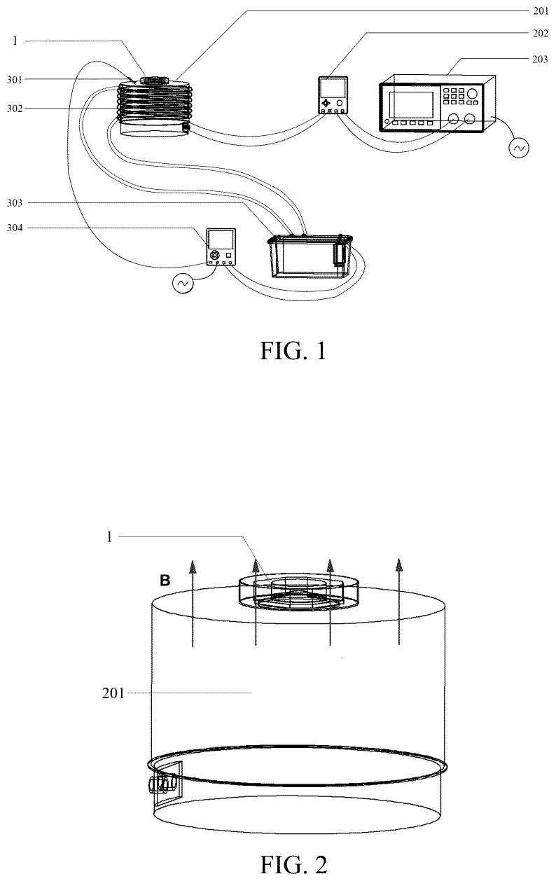

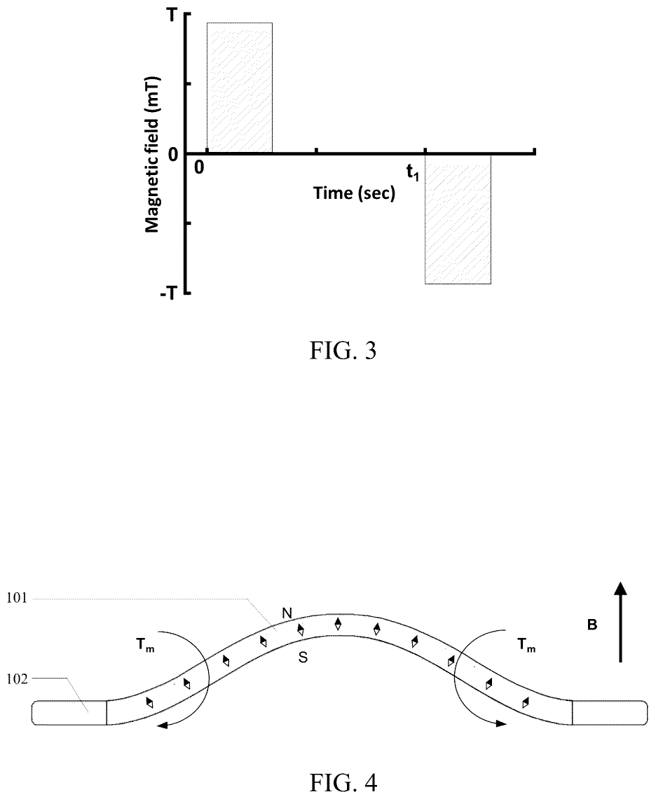

As shown in FIG. 1, the magnetic field generation module consists of an electromagnet 201, a current control unit 202, and a power supplier 203. The magnetic field generation module generates a direction-alternating magnetic field by switching the current direction. The magnet-driven bistable actuator is immersed in a Petri dish filled with the culture medium. It is positioned in the appropriate location above the pole of electromagnet 201, as depicted in FIG. 2.

The alternating current is generated by current control unit, yielding a direction-alternating magnetic field produced by the electromagnet. The direction-alternating magnetic field drives the magnetic bistable arched membrane to snap reversibly from concave mode to convex mode.

In the present disclosure, the magnet-driven bistable actuator, Petri dish, and electromagnet are placed in a CO₂ incubator.

As shown in FIG. 1, the temperature control module consists of a cooling system and the control circuit. The cooling system comprises a temperature sensor 301, a condensate tube 302, and a coolant tank 303. The temperature sensor detects the temperature of the cell culture medium surrounding the magnet-driven bistable actuator and transmits the temperature signal to the control circuit. The control circuit activates the cooling system to regulate and adjust the temperature of the electromagnet.

The condenser tube 302 surrounds the electromagnet 201 in the magnetic field generation module and is placed inside the CO₂ incubator along with the electromagnet. The control circuit 304 turns on the coolant tank 303 pump and regulates the flow rate of condensate liquid in the condenser tube. Ideally, the coolant tank 303 is a water tank.

Since the coil generates heat during the operation of the electromagnet 201, the pump switches on when the temperature sensor detects that the culture medium temperature is higher than the suitable temperature for cell survival. The condensate flow cools down the electromagnet via the condenser tube. The pump turns off the condensate flow as soon as the temperature drops below the suitable temperature. Preferably, 37° C.-39° C.

The magnetization direction of the magnet-sensitive bistable arch membrane is perpendicular to the tangent line of the membrane surface. The misalignment between the magnetization profile and the applied magnetic field introduces a magnetic moment to the magnet-driven actuator. This magnetic moment snaps the bistable arch membrane from a concave state to a convex state, and the reverse magnetic moment snaps the membrane back from the convex state to the concave state by alternating the direction of the magnetic field.

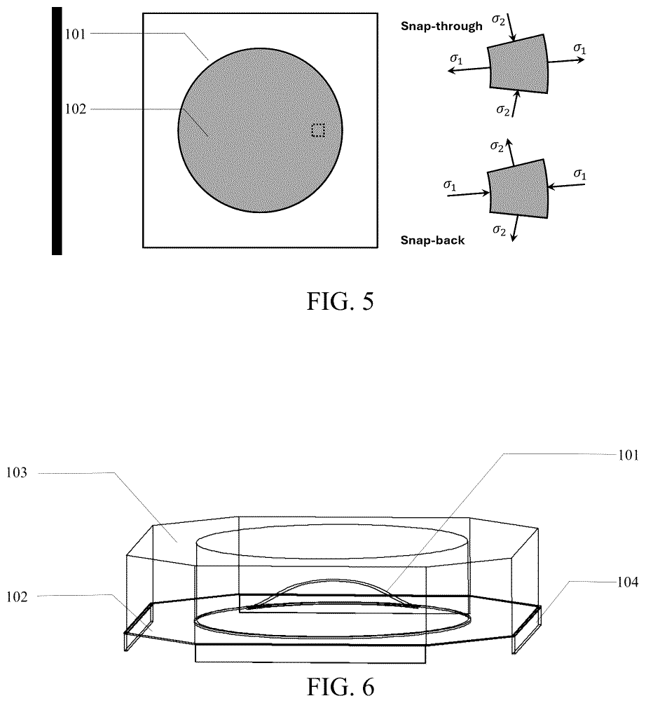

The bending deformation introduces radial and circumferential strains to the surface of the membrane during the snapping process, as illustrated in FIG. 5. These strains act on the cells cultured on the membrane, realizing biaxial and bidirectional loadings.

It is feasible to control the strains applied on cells by choosing bistable arch membranes with different deflections. Additionally, it is feasible to regulate the strain frequency by adjusting the magnetic field switching frequency.

Ideally, the deflection at the magnet-sensitive bistable arch membrane center is 2-3 mm. The thickness of the magnetic bistable arch membrane is 0.3-0.6 mm.

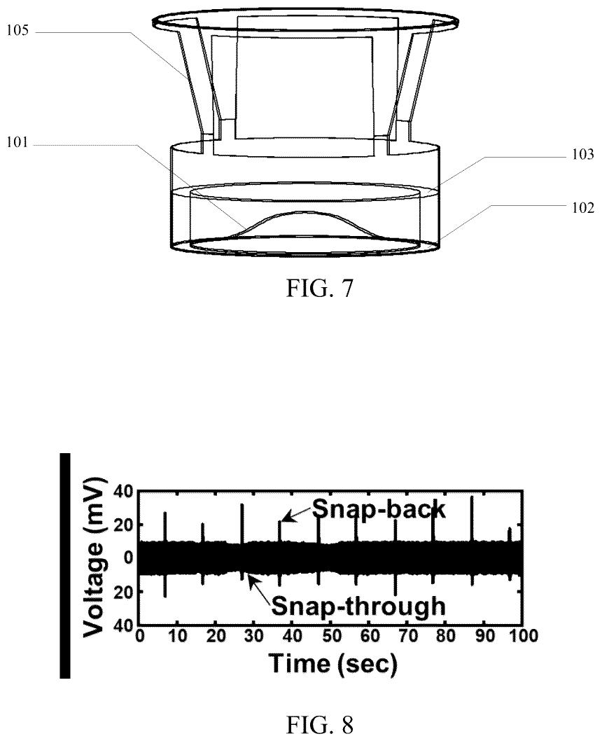

As shown in FIG. 4, the fixed periphery 102 bonds to the magnet-sensitive bistable arch membrane 101 circumferentially. For the first embodiment, the fixed periphery 102 bonds to the bracket frame 103 with the first support columns 104, as shown in FIG. 6. The fixed periphery constrains the rigid body displacement of the magnet-sensitive bistable arch membrane, while the support columns keep the membrane suspended in the cell culture medium. For the second embodiment, the fixed periphery 102 bonds to the bracket frame 103 and fixes in the bottom of the transwell insert, as shown in FIG. 7. The fixed periphery constrains the rigid body displacement of the magnet-sensitive bistable arch membrane, while the transwell insert keeps the membrane suspended in the cell culture medium.

In this disclosure, there are no restrictions on the specific shape of the bracket frame. For instance, it could be a column with an annular or polygonal base, such as an octagonal, square, or annular column, or something similar.

Specifically, the fixed periphery 102 has a circular hole adapted to fit the outer contour of the magnetic bistable arched membrane 101, and the circular hole is partially or wholly connected to the outer contour, limiting the rigid body displacement of the magnetic bistable arched membrane 101.

Preferably, the magnet-sensitive arch membrane 101 is bonded to the fixed periphery 102 by casting, and the fixed periphery 102 is bonded to the bracket frame 103 by using UV/ozone treatment.

As depicted in FIG. 6, for the first embodiment of the magnet-driven bistable actuator, the fixed periphery 102 is an octagonal sheet with a circular hole in the center, and the bracket frame 103 is a column with an octagonal base, bonded to the first support columns 104. The magnetic bistable arch membrane 101 is suspended in the culture medium.

As shown in FIG. 7, for the second embodiment of the magnet-driven bistable actuator, the fixed periphery 102 is a ring, and the bracket frame 103 is a cylinder. The fixed periphery 102 is bonded to the bottom of the cylinder. The magnet-driven bistable actuator is located on the bottom of the transwell insert and suspended in a culture medium.

Preferably, the magnetic bistable arch membrane has a three-dimensional curved structure, serving as a culture substrate for cells. In the present disclosure, the magnetic bistable arch membrane is made of biocompatible materials and magnetic particles. In detail, the biocompatible material comprises one or more of polydimethylsiloxane (PDMS), polyvinyl alcohol (PVA), polycaprolactone (PCL), gelatin methacrylate (GelMa), polyethylene terephthalate (PET), and polyurethanes (PU). The magnetic particles comprise at least one of rubidium-iron-boron (NdFeB) magnetic micro-particles, alnico magnetic particles, hard ferrite magnetic particles, samarium cobalt magnetic particles, and others. Specifically, the present disclosure also provides a method of preparing the magnet-driven bistable dynamic bioreactor, including the preparation method of magnet-sensitive arch membrane and a magnet-driven bistable actuator, the assembly method of the magnet-driven bistable actuator, the magnetic field generation module, and the temperature control module.

The preparation of magnet-sensitive bistable actuators includes:
1. cast a magnetic bistable arch membrane with PDMS/magnetic particles mixture;
2. reorientate the magnetization profile of the magnetic particles embedded in the magnet-sensitive bistable arch membrane by using a pulse magnetizer;
3. prepare a fixed periphery by 3D printing, and bond the fixed periphery to the bistable arch membrane;
4. cast a bracket frame with PDMS base liquid and crosslinking agent mixture.
5. bond the fixed periphery and support columns to the bracket frame.

Specifically, the preparation method of the magnet-sensitive bistable actuator includes:
1) inject PDMS/magnetic particles mixture into a mold with an arch-shaped cavity, degas in the mixture, and cast the magnet-sensitive bistable arch membrane in an oven;
2) reorient the magnetization profile of the arch membrane by applying a magnetization field;
3) 3D print a fixed periphery, and then bond the magnetic bistable arch membrane partially or wholly to the fixed periphery to constrain its displacement;
4) cast a cylinder with the same base shape as the fixed periphery, and bond the bracket frame to the fixed periphery by using plasma cleaner;
5) Outfit the bracket frame with support columns to guarantee the stable suspension of the magnet-sensitive bistable arch membrane in a culture medium.

Wherein the magnetic prepolymer mixture consists one of magnetic particles/polydimethylsiloxane (PDMS) mixture, magnetic particles/polyvinyl alcohol (PVA) mixture, magnetic particles/polycaprolactone (PCL) mixture, magnetic particles/acrylate gelatin methacrylate (Gel Ma) mixture, magnetic particles/polyethylene terephthalate (PET) mixture, and magnetic particles/polyurethanes (PU) mixture.

Specifically, the step 1) including:
Step 11) Mix the base liquid and crosslinking agent in a specific mass ratio to prepare the prepolymer mixture, then mix the prepolymer mixture with magnetic particles in a specific volume ratio to obtain the magnetic prepolymer mixture;
Step 12) Pour the magnetic prepolymer mixture into the mold, and remove air bubbles in a negative pressure oven;
Step 13) Cast the magnetic prepolymer mixture into the oven and de-mold it.

In one embodiment of the present invention, the magnetic particles are selected from one or more of the following options: the rubidium iron boron magnetic particles, alnico magnetic particles, hard ferrite magnetic particles, and samarium cobalt magnetic particles.

Preferably, the base liquid is mixed with the crosslinking agent in a mass ratio of 20:1 to 5:1, and the prepolymer mixture is mixed with the magnetic particles in a volume ratio of 10:5 to 2:1, For example, 10:1.

Preferably, the prepolymer mixture is polydimethylsiloxane (PDMS).

Preferably, magnetic prepolymer mixture is cast in an oven at 90-110° C. for 30-50 minutes in step 13).

As an example, the casting mold is an arch-shaped cavity with a diameter of 19 mm, a deflection of 2-3 mm, and a thickness of 0.5 mm.

Specifically, the step 2) includes:
Step 21) Flatten the magnetic bistable arch membrane with two clean glass slides;
Step 22) Push the flatten magnetic bistable arch membrane and the two glass slides down to the bottom of the magnetizer chamber;
Step 23) Reorient the magnetization direction of the magnetic particles by applying a magnetic field larger than the coercivity of the magnetic particles, aligning the magnetization of the particles perpendicular to the flatten membrane;
Step 24) Peel the flattened magnetic bistable arch membrane off the glass slide. After peeling, the magnetic bistable arch membrane reverts to its arch state, with the magnetization direction perpendicular to the tangent of the membrane surface.

As an example, the value of the magnetization field should be larger than the saturation magnetization of the chosen magnetic particles, such as from 1 T to 1.5 T.

In the above embodiment, reorientation magnetization of the arch membrane is a prerequisite for the magnetic bistable arch membrane to achieve configuration transition. The present disclosure provides two technical methods for magnetizing the magnetic bistable arch membrane: the magnetic particle magnetization method and the magnetic particle rearrangement method. The magnetic particle rearrangement method specifically applies a constant magnetic field to the magnetic bistable arch membrane during curing. This process arranges the magnetic particles in an orderly manner along magnetic field lines of the constant magnetic field, yielding an oriented magnetization pattern in the magnetic bistable arch membrane. This method involves magnetization and curing processes simultaneously. The magnetic particle magnetization method follows the curing of the magnetic bistable arch membrane. In this method, the membrane is first flattened, and then an instantaneous magnetic field, whose field strength this greater than the coercivity of the magnetic particles, is applied to the membrane. This magnetic field orientates the magnetic field of particles along the direction of the instantaneous magnetic field.

The magnetic particle rearrangement method is highly versatile, but it results in longer production cycles. On the other hand, the magnetic particle magnetization method is more convenient and faster, however, the size of the membrane capacity is restricted by that of the magnetizer chamber. Specifically, the preparation method of the fixed periphery in step 3) includes:
Step 31) Mix the base liquid and crosslinking agent in some mass ratio thoroughly to prepare the 3D printing ink;
Step 32) Print a fixed periphery using as-prepared 3D printing ink, there is a hole in the fixed periphery whose diameter is equal to that of the magnetic bistable arch membrane.
Step 33) Bond the edge of the arch membrane to the edge of the fixed peripheral hole after 3D printing, and cure the combination in an oven at 70° C. for 40 mins.

Preferably, in step 31), the 3D printing base liquid is mixed with the crosslinking agent in a mass ratio of 20:1 to 5:1. For example, 10:1.

Specifically, the step 4) includes:
Step 41) Mix the base liquid and crosslinking agent in a certain mass ratio thoroughly and degas by standing still;
Step 42) Pour the prepolymer into the mold cavity and degas under negative pressure to ensure the even distribution of the prepolymer. The mold is designed as a three-dimensional cylindrical structure, with a bottom surface shape consistent with that of the fixed periphery;
Step 43) Place the mold into an oven to cure the prepolymer into a bracket frame at 70° C. for 4 hours;
Step 44) Bond the as-prepared fixed periphery to the surface of the bracket frame using the UV Ozone plasma.

In step 44), the mass ratio of the base liquid and cross-linking agent varies from 20:1 to 5:1. For example, 10:1.

In steps 11) and 41), the base liquid consists of polydimethylsiloxane base liquid (PDMS), vinyl alcohol (PVA), caprolactone (PCL), acylated gelatin methacrylate (Gel Ma), ethylene dicarboxylate terephthalate (PET), and polyurethanes (Polyurethanes). The crosslinking agent is selected from those corresponding to the base liquid.

The present disclosure also provides a method of using the magnet-driven bistable dynamic bioreactor, including:
S1, sterilize the magnet-driven actuator in a 70% alcohol bath for 24 hours and then expose it to ultraviolet light for 30 minutes before culturing cells.
S2, sterilize the Petri dish, electromagnet 201, temperature sensor 301, and condenser tube 302 with a 70% alcohol solution and then expose them to ultraviolet light for 30 minutes; S3, wash the magnet-sensitive arch membrane with phosphate-buffered saline (PBS) 3 times, then soak in fibronectin for 20 minutes;
S4, seed the cells on one surface of the magnet-sensitive arch membrane 101 at an appropriate density and culture the cells for 24 hours without any loading to ensure cell adhesion.
S5, turn on the magnetic field generation module and apply biaxial, bidirectional, and frequency-adjustable dynamic loading to the cells;
S6, turn on the temperature control module and ensure that the temperature remains within the range suitable for cell survival.

When the bioreactor is in operation, the magnetic field generation module generates a direction-alternating magnetic field by changing the direction of the current. The misalignment between the applied magnetic field and the magnetization profile of the membrane introduces a magnetic torque, causing the bistable actuator to snap reversibly from one state to another. As the magnet-sensitive bistable arch membrane snaps through the symmetrical plane, it generates radial and circumferential strains, thereby subjecting the cells cultured on the membrane to complex mode loading in radial and circumferential directions.

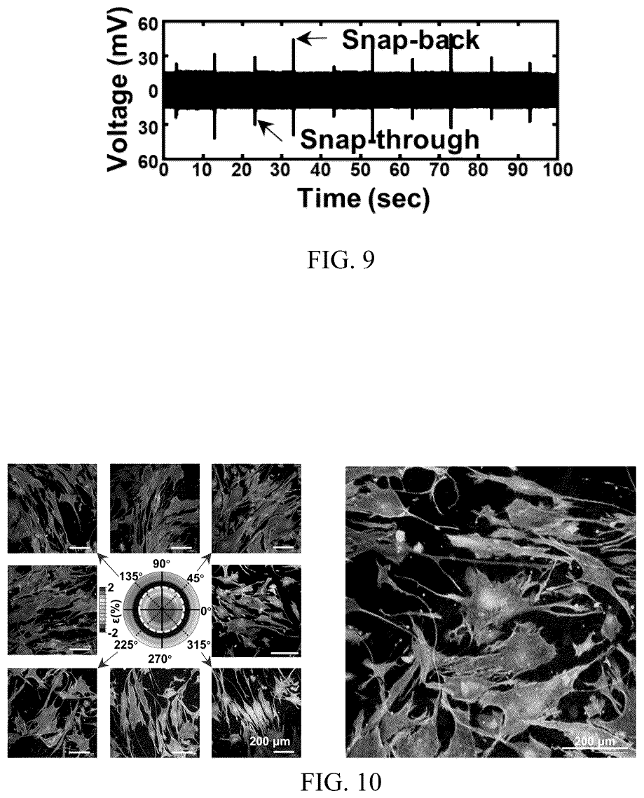

The magnet-driven bistable actuator 1 switches its configuration in the applied magnetic field. Cells cultured on the magnetic bistable arch membrane experience alternating radial strain (see FIG. 8) and circumferential strain (see FIG. 9). The snapping frequency ranges from 0.1 to 10 Hz.

Specifically, human dermal fibroblast cells are selected as an example to demonstrate the operational procedures of the dynamic bioreactor and to study the mechanical response of fibroblast cells to biaxial and bidirectional periodic loading.

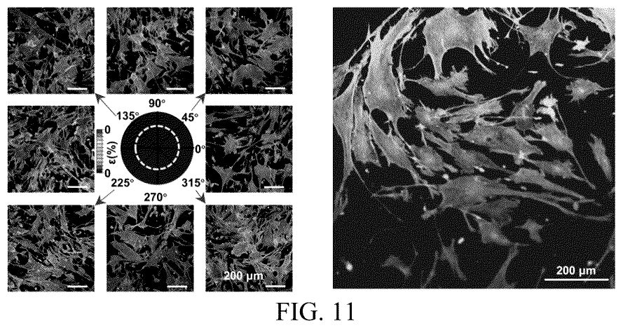

FIGS. 10 and 11 show the cytoskeleton and cell body reorientation of the experimental and control groups, respectively. To study the reorientation of human dermal fibroblast (HDFa) cells, each magnetic bistable arch membrane was divided into eight sectors 45° apart along the white ring shown in FIGS. 10 and 11 and one central section (right panel of FIG. 10 and right panel of FIG. 11). These areas were defined as analysis zones. The long axes of cells in the experimental group aligned along the radial direction in all peripheral analysis zones, in contrast to the random alignment observed in the control group.

In order to provide a clearer understanding of the objects, technical solutions, and advantages of the present disclosure, the following sections describe the present disclosure in further detail in connection with embodiments. It is important to emphasize that the specific embodiments described herein are intended to provide better elaboration of the present disclosure and are part of the embodiments of the present disclosure, but do not encompass all possible embodiments, and therefore do not serve to limit the present disclosure. Additionally, the technical features involved in the embodiments of the present disclosure described below may be combined as long as they do not conflict with each other.

### Embodiment 1

### Preparation of a Magnet-Driven Bistable Dynamic Bioreactor:

### (1) Preparation of Magnetic Bistable Arch Membrane.

Mix polydimethylsiloxane 184 base liquid and crosslinking agent at a mass ratio of 10:1, and then add the hard magnetic microparticles (NdFeB microparticles, with the particle size of 2-10 μm) to the liquid at the volume fraction of 5:1, yielding the PDMS/magnetic particles mixture. Pour some PDMS/magnetic particles mixture into the arch cavity (with a diameter of 19 mm, a deflection of 2-3 mm, a thickness of 0.5 mm), and remove air bubbles of the mixture under negative pressure conditions, and then cure polymer liquid in an oven at 90-110° C. for 30-50 minutes, and finally demold the membrane.

### (2) Reorientation of the Magnetic Profile of the Bistable Arch Membrane

Attach the as-prepared arch membrane to a clean glass slide, then cover it with another clean glass slide, pressing down to flatten the arch membrane. Subsequently, place the glass/membrane/glass combination at the bottom of the magnetizer chamber and reorient the magnetization profile by applying a magnetic field. Based on the hysteresis loop data of the selected magnetic particles, the value of the magnetization field should exceed the saturation magnetization of the chosen magnetic particles, such as 1.5 T. Remove the glass/membrane/glass combination from the plus magnetizer chamber, and peel the arch membrane off the glass slide. After reorientation, the magnetization profile is perpendicular to the tangent line of the arch membrane.

### (3) Fabrication of the Fixed Periphery

Mix polydimethylsiloxane 184 base liquid and crosslinking agent at a mass ratio of 10:1, then degas the mixture by using centrifuge to obtain 3D printing ink. The fixed periphery is designed as an octagonal shape (with a length of 12.43 mm and a thickness of 0.5 mm), embedding with a circle hole (with a diameter equal to the projection diameter of the arch membrane). The fixed periphery is fabricated by using a pneumatic extrusion direct-write 3D printer.

Bond the magnet-sensitive bistable arch membrane partially or completely along the circumference to the embedded circular hole of the uncured fixed periphery, then cure the assembly in an oven at 70° C. for 40 minutes. The circumference of the magnetic bistable arch membrane is constrained by the fixed periphery.

### (4) Fabrication of the Bracket Frame and the Assembly of the Magnetic Bistable Actuator.

Mix polydimethylsiloxane 184 base liquid and crosslinking agent at a mass ratio of 10:1 to yield the PDMS mixture. Pour some PDMS mixture into the casting mold. The bracket frame mold was designed as an octagonal column (with a side length of 12.43 mm and a thickness of 8 mm) embedded with a cylinder (with a diameter of 19). Remove air bubbles of the mixture under negative pressure conditions, and then cure polymer liquid in an oven at 90-110° C. for 30-50 minutes, and de-mold the cured bracket frame. Bond the fixed periphery to the bottom of the bracket frame by using plasma cleaner, and attach support columns to the side of the bracket frame to create the magnetic bistable actuator.

### Embodiment 2

The preparation method of embodiment 2 closely resembles that of embodiment 1, differing primarily in the shape of the fixed periphery and the bracket frame. The fixed periphery is fabricated as a cylinder tube with an outer diameter of 24 mm, an inner diameter of 20 mm, and a thickness of 0.5 mm, and the bracket frame is fabricated as a cylinder having an outer diameter of 24 mm, an inner diameter of 20 mm, and a depth of 8 mm. In the second embodiment of the magnet-driven bistable actuator, it is pushed to the bottom of the transwell insert, specifically the second support column 105, allowing the assembly to suspend in the culture medium.

The foregoing is only a preferred application embodiment of the present disclosure, and it should be noted that for a person of ordinary skill in the art, several improvements and embellishments can be made without departing from the technical principles of the present disclosure, and these improvements and embellishments should also be regarded as the scope of protection of the present disclosure.

1. A magnet-driven bistable dynamic bioreactor comprises:
one magnet-driven actuator,
which includes a magnet-sensitive arch membrane, a fixed periphery, a bracket frame, and supporting component wherein the magnet-sensitive arch membrane is capable of snapping through and snapping back by application of magnetic field, enabling cell cultivation;
a magnetic field generation module,
wherein the magnetic field generation module provides a magnet field to drive the actuator;
and a temperature control module;
wherein the temperature control module adjusts the temperature of the culture medium.

2. The magnet-driven bistable dynamic bioreactor in claim 1, wherein the magnet-sensitive arch membrane is capable of maintaining two stable configurations without any loading and switching these configurations with external loading.

3. The magnet-driven bistable dynamic bioreactor in claim 1, wherein the magnet-sensitive arch membrane comprises at least one hard magnetic particle to introduce the magnetic profile.

4. The magnet-driven bistable dynamic bioreactor in claim 1, wherein the magnetic profile of the magnet-sensitive arch membrane introduces an alternative magnetic direction torque to snap the membrane through and back.

5. The magnet-driven bistable dynamic bioreactor in claim 1, wherein the fixed periphery defines a circular hole to bond the magnetic bistable arch membrane and to constrain its displacement during snapping, thereby generating the strain field on the surface of the membrane.

6. The magnet-driven bistable dynamic bioreactor in claim 1, wherein the bracket frame defines a cylinder with a hollow tube, configured to provide actuation space for the snapping process, and to hold the arch membrane in the culture medium during snapping.

7. The magnet-driven bistable dynamic bioreactor in claim 1, wherein the supporting component in embodiment 1 defines at least three columns bonded to the bottom of the bracket frame, configured to hold the magnet-driven actuator in culture medium.

8. The magnet-driven bistable dynamic bioreactor in claim 1, wherein the supporting component in embodiment 2 defines a transwell insert to hold the magnet-driven actuator in the culture medium.

9. The magnet-driven bistable dynamic bioreactor in claim 1, wherein the cross-section of the bracket frame is similar to that of the fixed periphery, enabling them to be bonded together.

10. The magnet-driven bistable dynamic bioreactor in claim 1, wherein the magnet-sensitive arch membrane, the fixed periphery, the bracket frame, and the supporting component are made of biocompatible polymer for cell viability, proliferation, and differentiation.

11. The magnet-driven bistable dynamic bioreactor in claim 1, wherein the magnet-driven actuator and the dish define a well to hold enough culture medium for cell viability, proliferation, and differentiation.

12. The magnet-driven bistable dynamic bioreactor in claim 1, wherein the magnetic field generation module comprises:
an electromagnet;
a current control unit, wherein the current control unit switches the direction current of the electromagnet, thereby changing the direction of the magnetic field cyclically;
and a power supply.

13. The magnet-driven bistable dynamic bioreactor in claim 1, wherein the temperature control module comprises:
a temperature sensor for detecting the temperature of the culture medium;
a condenser tube; a liquid tank and a control circuit.

14. The magnet-driven bistable dynamic bioreactor in claim 13, wherein the condense tube defines a helical pipe wrapped around the electromagnet, configured to reduce its temperature to the pre-set value.

15. The magnet-driven bistable dynamic bioreactor in claim 13, wherein the control circuit is configured to control the flow rate of the condensate in the condense tube.

16. A method of preparing the magnet-driven bistable dynamic bioreactor according to claim 1 involves the following steps:
preparing a magnet-driven bistable actuator, then connecting the magnet-driven bistable actuator to a magnetic field generation module and a temperature control module.
The method of preparing magnet-driven bistable actuator includes:
creating a magnetic bistable arch membrane (101) containing magnetic particles using a casting method;
employing the magnetic particle magnetization method to magnetize the magnetic bistable arch membrane (101) in a specific direction;
producing a fixed periphery (102) with a hole tailored to the magnetic bistable arch membrane using a 3D printing process and bonding the fixed periphery (102) to the magnetic bistable arch membrane;
affixing the fixed periphery (102) at the corresponding position of the bracket frame (103) and constructing a support column for stable placement of the fixed periphery (102) in the cell culture medium.

17. The preparing method according to claim 16 includes:
1) injecting a magnetic prepolymer mixture into a mold with an arch cavity using the pouring method, and producing a magnetic bistable arch membrane (101) through degassing, drying and demolding steps;
2) magnetizing the magnetic bistable arch membrane (101) by applying an instantaneous magnetic field magnetic field based on the hysteresis loop property of the magnetic particles, orienting the magnetic profile in the arch membrane (101) perpendicular to the tangent line of the surface of the arch membrane, and obtaining a magnetized magnetic bistable arch membrane (101);
3) preparing a fixed periphery (102) and bonding the magnetic bistable arch membrane (101) to the fixed periphery, either partially or entirely, to confine the magnetic bistable arch membrane;
4) creating a bracket frame (103) and establishing a permanent interlayer bonding between the bracket frame (103) and the fixed periphery (102);
5) attaching a support column to the bracket frame (103) to ensure the stable placement of the magnetic bistable arch membrane in the cell culture medium.

18. The application of the magnet-driven bistable dynamic bioreactor according to claim 1 involves simulating a real stress state of human cells in their internal environment, such as, human dermal fibroblasts, human epidermal keratinized cells, renal distal tubular epithelial cells, human cardiomyocytes, osteoblasts and the like.
